(19) United States
(12) Patent Application Publication
(10) Pub. No.: US 2008/0160097 A1
(43) Pub. Date: Jul. 3, 2008

(54) Method for imparting a cosmetic or therapeutic benefit to a topical surface and process for making

(75) Inventors: Ernest Curtis, Milford, PA (US); Steven E. Brown, New Windsor, NY (US); Tameru Getahoun, Parsippany, NJ (US)

Correspondence Address: Charles N. J. Ruggiero;Ohlandt, Greeley, Ruggiero & Perle, L.L.P., 10th Floor, One Landmark Square, Stamford, CT, 06901-2682, US

(73) Assignee: AVON PRODUCTS, INC.

(21) Appl. No.: 11/646,759

(22) Filed: Dec. 28, 2006

(51) Int. Cl. A61K 9/14

(52) U.S. Cl. 424/501

(57) ABSTRACT

There is disclosed a method of imparting a cosmetic or therapeutic benefit to a topical surface. The method has the steps of a) providing a polymer powder having a plurality of substantially nonporous polymer particles with a number average particles size of about 0.1 to about 0.5 microns in diameter; b) providing a liquid cosmetic or therapeutic composition in the form selected from the group consisting of a solution, dispersion, suspension, and an emulsion in which the liquid composition has one or more cosmetic or therapeutic actives therein and is metastable; c) contacting the polymer powder with the liquid composition so that the liquid composition is entrained so as to form an impregnated powder; and d) rubbing the impregnated powder on the topical surface such that at least a substantial portion of the liquid composition is released from the impregnated powder at the topical surface. There are other methods and processes disclosed.

### BACKGROUND OF THE INVENTION

1. Field of the Invention

The invention relates a method for imparting a cosmetic or therapeutic benefit to a topical surface. The invention further relates to a process for making an impregnated powder for imparting a cosmetic or therapeutic benefit to a topical surface.

2. Description of the Related Art

Face powder cosmetics are primarily used to absorb excess makeup and to absorb oil and sebum secreted by the skin. Typically, powders are used in conjunction with liquid-based foundations to obtain longer wear from the liquid composition, known as “setting”, and further to provide a soft focus, matte finish appearance to the skin. Likewise, powders can be used alone to control shine and impart a cosmetic effect on the skin. In certain applications, powder compositions are preferred to liquid compositions due to their light feel and ease of use in achieving a desired appearance.

A difficulty encountered when using powder cosmetics alone is that they do not provide long wear and tend to exhibit fading and creasing. Powder cosmetics also do not resist transfer once applied to the skin. In addition, powders cosmetics can dehydrate the skin and require the user to seek alternate means to moisturize the skin. In contrast, liquid foundations, while having superior wear and transfer resistance, may feel heavy on the skin and require more skill during application than powder cosmetics to achieve a desired appearance.

Conventional powders, such as talcum and corn starch, have been used to carry liquids as cosmetic or therapeutic actives or carriers for same. Such powders are very absorptive/adsorptive but have limited capability to express or expel liquids on application of the powders to a topical surface. Furthermore, some starch-based materials are known to chemically interact with aqueous media to form gels.

Other powders used to carry liquids as cosmetic or therapeutic actives or carriers for same are of the powder-to-liquid-to-powder type. Liquids are entrained or encapsulated in powders for subsequent topical application. Such powders are disclosed, for example, in U.S. Patent Application No. 2006/0008485 A1.

Water-in-oil, water-in-silicone, and water-in-wax emulsions are commonly employed in cosmetic and therapeutic skin care products. End users particularly enjoy the soft, smooth feel imparted by silicones. Water is used in such emulsions to lower viscosity, reduce the greasy feel normally associated with some of non-aqueous components, and to carry hydrophilic adjuvants. Making products with such emulsions has been difficult due to high levels of emulsifier required to stabilize such products. High levels of emulsifier can cause skin irritation and sensitization in end users and reduce wear performance of the cosmetic composition.

It would be desirable to have a method for imparting a cosmetic or therapeutic benefit to a topical surface with a powder that afforded the benefits of both powder and liquids. It would be further desirable to have a method for imparting a cosmetic or therapeutic benefit with a powder-to-liquid-to-powder composition that afforded superior delivery, functionality, and transfer performance. It would still be further desirable to have a process for making an impregnated powder for imparting a cosmetic or therapeutic benefit to a topical surface. It would yet be further desirable to have cosmetic and therapeutic skin care products that can be more effectively formulated as a water-in-oil, water-in-silicon, or water-in-wax emulsion and be less irritating to end users.

### SUMMARY OF THE INVENTION

There is provided a method for imparting a cosmetic or therapeutic benefit to a topical surface.

There is provided a method for imparting a cosmetic or therapeutic benefit to a topical surface with a polymer powder that it adapted to deliver a substantial amount of liquid.

There is provided a process for making an impregnated powder for imparting a cosmetic or therapeutic benefit to a topical surface.

There is provided a method of imparting a cosmetic or therapeutic benefit to a topical surface. The method has the steps of a) providing a polymer powder having a plurality of substantially nonporous polymer particles having a number average particles size of about 0.1 to about 0.5 microns in diameter; b) providing a liquid cosmetic or therapeutic composition in the form selected from the group consisting of a solution, dispersion, suspension, and an emulsion in which the liquid composition has one or more cosmetic or therapeutic actives therein and is metastable; c) contacting the polymer powder with the liquid composition so that the liquid composition is entrained so as to form an impregnated powder; and d) rubbing the impregnated powder on the topical surface such that at least a substantial portion of the liquid composition is released from the impregnated powder at the topical surface.

There is provided another method of imparting a cosmetic or therapeutic benefit to a topical surface. The method has the following steps: a) providing a polymer powder having a plurality of substantially nonporous polymer particles with a number average particles size of about 0.1 to about 0.5 microns in diameter; b) providing a liquid cosmetic or therapeutic composition selected from the group consisting of an water-in-oil emulsion, water-in-silicon emulsion, and a water-in-wax emulsion in which the liquid composition has one or more cosmetic or therapeutic actives; c) contacting the polymer powder with the liquid composition so that the liquid composition is entrained so as to form an impregnated powder; and d) rubbing the impregnated powder on the topical surface such that at least a substantial portion of the liquid composition is released from the impregnated powder at the topical surface.

There is provided another method of imparting a cosmetic or therapeutic benefit to a topical surface. The method has the following steps: a) providing a polymer powder having a plurality of substantially nonporous polymer particles having a number average particles size of about 0.1 to about 0.5 microns in diameter; b) providing two or more reactive components in liquid form therein that are reactable with each other to form one or more cosmetic or therapeutic actives or produce one or more cosmetic or therapeutic effects; c) contacting each of the two or more reactive components in liquid form with a batch polymer powder to form two or more impregnated batch polymer powders in which each reactive component is entrained in an impregnated batch powder; d) admixing the two or more impregnated batch polymer powders to form a combined impregnated polymer powder; and e) rubbing the combined impregnated powder on the topical surface such that at least a substantial portion of the two or more reactive components are released from the combined impregnated powder and are allowed to react to produce the one or more cosmetic or therapeutic actives or the one or more cosmetic or therapeutic effects at the topical surface.

There is also provided another method of imparting a cosmetic or therapeutic benefit to a topical surface. The steps of the method are the following: a) providing a polymer powder having a plurality of substantially nonporous polymer particles having a number average particles size of about 0.1 to about 0.5 microns in diameter; b) providing two or more incompatible cosmetic or therapeutic components in liquid form; c) contacting each of the two or more incompatible components in liquid form with a batch polymer powder to form two or more impregnated batch polymer powders in which each incompatible component is entrained in an impregnated batch powder; d) admixing the two or more impregnated batch polymer powders to form a combined impregnated polymer powder; and e) rubbing the combined impregnated powder on the topical surface such that at least a substantial portion of the two or more incompatible components are released at the topical surface.

There is provided a process for making an impregnated polymer powder for imparting a cosmetic or therapeutic benefit to a topical surface. The process has the following steps: a) providing a polymer powder having a plurality of substantially nonporous polymer particles having a number average particles size of about 0.1 to about 0.5 microns in diameter; b) providing a liquid cosmetic or therapeutic composition in the form selected from the group consisting of a solution, dispersion, suspension, and an emulsion wherein the liquid composition has one or more cosmetic or therapeutic actives therein and is metastable; and c) contacting the polymer powder with the liquid composition so that the liquid composition is entrained so as to form an impregnated powder.

There is provided another process for making an impregnated polymer powder for imparting a cosmetic or therapeutic benefit to a topical surface. The process has the following steps: a) providing a polymer powder having a plurality of substantially nonporous polymer particles with a number average particles size of about 0.1 to about 0.5 microns in diameter; b) providing a liquid cosmetic or therapeutic composition selected from the group consisting of an water-in-oil emulsion, water-in-silicon emulsion, and a water-in-wax emulsion in which the liquid composition has one or more cosmetic or therapeutic actives; and c) contacting the polymer powder with the liquid composition so that the liquid composition is entrained so as to form an impregnated powder.

There is provided another process for making an impregnated polymer powder for imparting a cosmetic or therapeutic benefit to a topical surface. The process has the following steps: a) providing a polymer powder having a plurality of substantially nonporous polymer particles having a number average particles size of about 0.1 to about 0.5 microns in diameter; b) providing two or more reactive components in liquid form therein that are reactable with each other to form one or more cosmetic or therapeutic actives or produce one or more cosmetic or therapeutic effects; c) contacting each of the two or more reactive components in liquid form with a batch polymer powder to form two or more impregnated batch polymer powders in which each reactive component is entrained in an impregnated batch powder; and d) admixing the two or more impregnated batch polymer powders to form a combined impregnated polymer powder.

There is provided another process for making an impregnated polymer powder for imparting a cosmetic or therapeutic benefit to a topical surface. The process has the following steps: a) providing a polymer powder having a plurality of substantially nonporous polymer particles having a number average particles size of about 0.1 to about 0.5 microns in diameter; b) providing two or more incompatible cosmetic or therapeutic components in liquid form; c) contacting each of the two or more incompatible components in liquid form with a batch polymer powder to form two or more impregnated batch polymer powders in which each incompatible component is entrained in an impregnated batch powder; and d) admixing the two or more impregnated batch polymer powders to form a combined impregnated polymer powder.

### DETAILED DESCRIPTION OF THE INVENTION

The polymer powder useful in the present disclosure is generally formed by the following steps of (a) homogeneously mixing a solution having from 0.1 parts by weight to less than 25 parts by weight of a monomer selected from one or more types of polyunsaturated monomer, from greater than 75 parts by weight up to 99.9 parts by weight of a monomer solvent wherein the total weight of the monomers and the monomer solvent is 100 parts, and 0.05 to 5 weight percent initiator based on the weight of said monomer, and (b) polymerizing the monomers to form a polymer wherein the monomer solvent is a solvent for the monomers and a non-swelling non-solvent for the polymer. The resulting polymer powder can adsorb large quantities of liquids and substances admixed or dispersed therein.

Useful monomers have at least two unsaturated bonds (hereinafter referred to as “polyunsaturated” monomers) and containing no comonomers having a monounsaturated moiety. Examples include poly-acrylates (“poly” meaning two or more), -methacrylates, or -itaconates of ethylene glycol, propylene glycol, di-, tri-, tetra-, or poly-ethylene glycol and propylene glycol, trimethylol propane, glycerine, erythritol, xylitol, penta erythritol, di penta erythritol, sorbitol, mannitol, glucose, sucrose, cellulose, hydroxyl cellulose, methyl cellulose, 1,2 or 1,3 propanediol, 1,3 or 1,4 butanediol, 1,6 hexanediol, 1,8 octanediol, cyclohexanediol, or cyclohexanetriol. Others include bis(acrylamido or methacrylamido) compounds. Still other monomers include represented by di or poly vinyl esters such as divinyl-oxalate, -malonate, -succinate, -glutamate, -adipate, -sebacate, -maleate, -fumarate, -citraconate, and mesaconate. Still yet other monomers are represented by di or poly vinyl ethers of ethylene, propylene, butylene, glycols, glycerine, pentaerythritol, and sorbitol.

A single monomer or mixtures of two or more different monomers can be used to polymerize the porous powders. Monomers may be selected according to the adsorption properties desired. The chemical structure and adsorption property can be adjusted via changing the structure of the chain between the unsaturated bonds. For example, if the monomer includes a saturated hydrocarbon structure like 1,4 butanediol bis methacrylate, the adsorptivity of the powder will be shifted more to the hydrophobic side, while tetraethylene glycol bis methacrylate, having —CH₂CH₂O—units in its connecting chain, will impart a more hydrophilic character.

Polymerization is effected by dissolving the monomers or their mixtures in a solvent that does not swell or dissolve the resulting polymer. Based on the parts by weight of the monomer and the solvent totaling 100 parts by weight, the monomers are used from 0.1 to less than 25 parts by weight, preferably, from 2 to less than 25 parts by weight, and, more preferably, from 5 to 20 parts by weight. Correspondingly, the solvent is present from greater than 75 parts by weight to 99.9 parts by weight, preferably, from greater than 75 parts by weight to 98 parts by weight, and, most preferably, from 80 parts by weight to 95 parts by weight. In most instances, alcohols can be used as the monomer solvent. Preferably, the solvent is relatively volatile, having a boiling point of less than 80° C. at one atmosphere and is water-miscible. Removal of the solvent may be effected by filtration and evaporation, e.g., by heat and/or vacuum.

Polymerization is effected by using a free radical initiator, such as an azo compound, a peroxy dicarbonate, a peroxy ester, or a sulfonyl acid peroxide. Other useful initiators are sodium, potassium, or ammonium persulfates. Preferred initiators are redox initiators, such as secondary or tertiary amines. A more preferred initiator is a tertiary amine and peroxide combination. Preferably, the free radical initiator will have a 10-hour half-life temperature of 75° C. or less, i.e., it is a low to medium temperature initiator. The initiator is employed in an amount from 0.05 to 5 weight percent of the total monomer charge.

Polymerization is carried out in the presence of an inert atmosphere. This condition may be achieved by the use of a headspace gas such as nitrogen, argon, carbon dioxide, and the like. Typically, no stirring or very slow stirring (e.g., zero to 300 rotations per minute) is employed.

The reaction is maintained for such time as is required to achieve the desired yield of polymer. This time may be as little as one half-hour. However, to approach theoretical yield, 24 to 48 hours at room temperature, or 4 to 10 hours at elevated temperatures, are required. The monomer solvent is subsequently removed by filtration and evaporation resulting in a dry powder, which can be post-adsorbed with a variety of cosmetic active ingredients.

Additional teachings to the polymer powders and their manufacture are disclosed in U.S. Pat. Nos. 4,962,133; 4,948,170; and 4,962,170, which are incorporated herein by reference. Preferred finely divided polymer powders are lauryl methacrylate/dimethacrylate crosspolymer and sold under the trade name Polytrap (Amcol Health & Beauty Solutions).

The polymer powder is a combined system of particles. While the particles are substantially nonporous, the combined system takes the form of a porous, finely divided, free-flowing powder. The system of particles includes submicron unit particles ranging in size from 0.1 to 0.5 microns in diameter. The particles may range from elliptical to spherical in shape. A typical diameter of a particle is about 0.3 microns. The powder also consists of agglomerates of fused unit particles of sizes in the range of about ten (10) to eighty (80) microns in average diameter, and aggregates of clusters of fused agglomerates of sizes in the range of about two hundred (200) to about eight hundred (800) microns in average diameter. When mild pressure is applied to the powder, the aggregates and agglomerates are easily crushed into the small particles. Thus, the powder can be described as being “soft” in that the aggregate and agglomerate structure easily changes upon pressure and cannot be easily felt when rubbed on a topical surface such as the skin. The powders substantially disappear when rubbed upon a topical surface. Though not bound by any theory, disappearance is believed to result from the scission of aggregates to smaller size. The smaller aggregates approach the wavelength of visible light in size and appear to disappear to the eye. The powders do not swell in common solvents and are capable of physically adsorbing active ingredients by filling of interstitial voids via capillary action. The powders are often capable of adsorbing from sixty to ninety weight percent of a liquid based on the weight of the impregnated powder (powder plus liquid and adjuvants) and yet remain free flowing. The unit particles themselves do not have any significant porosity, usually less than 2%.

The size and adsorptive properties of the polymer powders can be influenced by the concentration of monomers and stirring rate. Lower concentrations of monomers and slower stirring typically result in powders of higher adsorptivity. Monomers can also be selected to enhance water and/or oil and silicone adsorption. For instance, relatively slow reaction stirring rates during the precipitation polymerization process produce highly structured particles that are capable of entraining significant quantities of liquid. Spherical particles, while within the scope of the present disclosure, are less structured and not capable of entraining as much liquid as highly structured particles.

The nature of the polymer powder permits it to adsorb liquids and cosmetic actives so as to function as a carrier or delivery system to permit controlled application to a topical surface. The polymer powder is particularly useful in a powder-to-liquid-to-powder system. In a powder-to-liquid-to-powder system, the polymer powder is contacted, i.e., mixed, with a liquid to form an impregnated powder, which takes a substantially powder-like form like a dry powder and is free-flowing like a dry powder. The impregnated powder is then topically applied with sufficient pressure or shearing, i.e., rubbing, to express at least a substantial or majority portion of and preferably substantially all of the adsorbed liquid from the impregnated powder. The liquid remains on the topical surface until volatile components evaporate to leave a powder residue having the cosmetic or therapeutic active therein. The impregnated powder exhibits the performance attributes of both liquid and powder-based cosmetic compositions.

The polymer powder preferably absorbs up to about 90 wt % of a liquid composition (liquid(s) and cosmetic/therapeutic liquids therein) based on the total weight of the powder. Preferably, the powder absorbs from about 50 wt % to about 90 wt % of a liquid composition based on the total weight of the powder. Most preferably, the powder preferably absorbs about 75 wt % to about 88 wt % of a liquid composition based on the total weight of the powder.

Another aspect of the method of the present disclosure is the ability to use and stabilize a metastable liquid cosmetic composition, particularly in metastable mixtures in the form of solutions, dispersions, suspensions, or emulsions. A metastable composition is a composition that is phase stable in its liquid state at ambient conditions for less than 3 months. The present disclosure is particularly useful with a metastable composition since it can be contacted with the polymer powder prior to loss of phase stability. The adsorption of the metastable composition into the powder effectively stabilizes it for later delivery to a topical surface. The ability to effectively stabilize a metastable liquid cosmetic composition is useful because it provides greater flexibility in formulation of the liquid composition.

Another aspect of the present disclosure is the powder-to-liquid-to-powder functionality. The polymer powder, in addition to providing a means of delivering liquid compositions, imparts benefits typically associated with powder compositions, e.g., impartation of a matte finish, ease of spreading, and improved wear characteristics.

An embodiment of the method of the present disclosure is the ability to use and stabilize incompatible ingredients or components in liquid cosmetic compositions, particularly incompatible mixtures in the form of solutions, dispersions, suspensions, and emulsions. The polymer powder provides a physical particle barrier between incompatible ingredients or components. Incompatible components are those ingredients in which physical mixtures are substantially immiscible or exhibit some other nonreactive negative or incompatible effect. An example of incompatibility is the combination of Carbopol gels and high levels, e.g., about or above 2 wt %, of pigments. Carbopol gels can cause pigments to flocculate. In that instance, the Carbopol gels and the pigments are adsorbed into separate batches of powder and the batch powders admixed for subsequent delivery to a topical surface. Another example of incompatibility is that of Carbopol gels and an acidic environment (pH less than about 5). Acidic environments cause Carbopol gels to break down and lose their gellation. In that instance, the Carbopol gels and the low pH components are adsorbed into separate batches of powder and the batch powders admixed for subsequent delivery to a topical surface.

Another aspect of the method of the present disclosure is the ability to use and stabilize two or more chemically reactive ingredients or components in the impregnated powder for subsequent delivery to the surface of the skin. Reactive ingredients can be liquids, semisolids, or solids. In the instance of solids, the solid is either melted or dissolved/dispersed/suspended/emulsified in a liquid solvent or vehicle or carrier for subsequent adsorption into the polymer powder. Reactive liquids or liquids with reactive ingredients therein can take any known form, such as solution, dispersion, suspension, or emulsion and can be stable or metastable. The liquids are subsequently adsorbed into the polymer powder. Each reactant will be adsorbed into a separate batch of polymer powder. Then the batches will be admixed, preferably by non-shearing blending with a ribbon blender or a tumbler. When the impregnated powder (having adsorbed reactants therein) is rubbed on the topical surface such that reactive components are released from the impregnated powder, the reactive ingredients will chemically react at the topical surface to produce a desired cosmetic or therapeutic active and/or effect or benefit. Possible reactions types include the following: acid-base reactions, reduction-oxidation reactions, polymerization reactions, thermal reactions, and photoactive reactions. An example of a reactive impregnated powder is one having calcium chloride and water in separate, entrained powder particles. When the impregnated powder is rubbed on the skin, the powder releases the calcium chloride and water, which react exothermically to produce a sensation of heating on the skin.

One embodiment of the method of the present disclosure is the topical application of a polymer powder having an emulsion therein. Useful emulsions include oil-in-water emulsions, water-in-oil emulsions, silicone-in-water emulsions, water-in-silicone emulsions, and triple emulsions. Further, lipophilic waxes can be added to or substituted for the oil or the silicone in the nonaqueous phase. Emulsions can be prepared by any means known in the art. Typically, the aqueous phase and the silicone phase or oil phase are separately prepared. One or more emulsifiers are added to any or all of the phases and the phases blended to form the emulsion.

Useful silicone oils and elastomers include dimethicones (such as Dow Corning DC556), polyorganosiloxanes (such as Dow Corning DC200), silicone siloxanes (such as General Electric SF series blends), dimethiconols (such as BASF Masil SFR series), silicone elastomer blends (such as Dow Corning 9040), cyclopentasiloxane, PEG-10 dimethicone and methicone, fluorosilicones, and phenyldimethicones. Useful oil phase liquids include vegetable oils, fatty acid esters, fatty alcohols, mineral oils, and hydrocarbon oils. Useful amphiphilic liquids include monohydric alcohols and polyhydric alcohols. Monohydric alcohols include those of 1 to 9 carbon atoms, such as ethanol, n-propanol, isopropanol, n-butanol, and n-pentanol. Polyhydric alcohols include glycerin, ethylene glycol, propylene glycol, butylene glycol, pentylene glycol, hexylene glycol, and oligomeric and polymeric forms thereof.

Emulsions commonly have from about 10 wt % to about 90 wt %, more commonly about 30 wt % to about 70 wt %, and still more commonly about 40 wt % to about 60 wt % water based on the total weight of the impregnated powder. Useful emulsifiers include sorbitan esters, glycerol esters, polyoxyethylene fatty esters and ethers, and PEG/PPG dimethicones. Additional emulsifiers are disclosed in Table 1 of U.S. Pat. No. 5,244,665, which is incorporated herein by reference. The sorbitan esters and glycerol esters are particularly useful in formulating water-in-oil, water-in-silicon, and water-in-wax emulsions (water-in-oil/silicone/wax emulsions). Emulsifiers for water-in-oil/silicone/wax emulsions typically range from 1 to 6 and above that range for oil/silicone/wax-in-water emulsions. Emulsions useful in the present disclosure for adsorption into polymer powders include stable and metastable emulsions.

The present disclosure is particularly useful in delivering cosmetic and therapeutic water-in-oil/silicone/wax emulsions to the skin. The present disclosure allows such emulsions to be formulated as metastable and be subsequently stabilized via adsorption into the polymer powder. Being formulated as a metastable emulsion affords greater flexibility in selection of ingredients, including emulsifier. Greater flexibility is afforded in both selection and amount of emulsifier. The same flexibility is likewise afforded to selection of other cosmetic and therapeutic ingredients and vehicles/carriers.

The polymer powder can have any known cosmetic or therapeutic agent adsorbed therein, such as the following: anesthetics, anti-allergenics, antifungals, antimicrobials, anti-inflammatory agents, antioxidants, antiseptics, chelating agents, colorants, depigmenting agents, emollients, emulsifiers, exfollients, film formers, fragrances, humectants, insect repellents, lubricants, moisturizers, photostabilizing agents, preservatives, skin protectants, skin penetration enhancers, sunscreens, stabilizers, surfactants, thickeners, viscosity modifiers, vitamins, or any combinations thereof. Cosmetic or therapeutic agents that are in solid form are either melted or incorporated into a solvent or liquid vehicle/carrier prior to adsorption into the polymer powder.

Examples of useful cosmetic products that may utilize the polymer powder include blushes, foundations, concealers, eyeshadows, mascaras, skin care, lip compositions, hair care, personal care, nail compositions. Examples of useful skin products that may utilize the polymer powder include skin conditioners, moisturizers, whiteners, anesthetics, dewrinkling compositions, sunscreens, insect repellents, self-tanning compositions.

In the present disclosure, the term “topical surface(s)” refers to skin, hair, lips, and nail. Preferred applications of the present invention are directed to the skin.

### EXAMPLES

Water-in-oil emulsions in the Examples are generally formed in the following manner. Non-aqueous components are placed in a 1-liter beaker and subsequently heated to 140° F. using a hot plate. A homogenizer (Silverson L4RT) equipped with a high-speed homogenization head (¾ tubular type impeller using an emulsifier screen) is used to mix the non aqueous composition at 3600 rpm. The components of the aqueous phase are added in a separate 1-liter beaker and thoroughly mixed prior to adding to the oil phase composition at 120F. The aqueous phase is added slowly under high shear mixing (greater than 5000 rpm) and allowed to mix for 30 minutes once at 120° F. The emulsion is allowed to cool to room temperature under low shear, 3000 rpm.

### Example 1

A powder-to-liquid-to-powder composition having a metastable emulsion with cosmetic actives was prepared. 150 grams of Part B, the lauryl methacrylate/dimethacrylate crosspolymer particle, is placed into a large stainless steel mixing bowl suitable or use with a Hobart Mixer (Model A-120) equipped with a wire-whisk mixing implement. Under low mixing 850 grams of emulsion Part A is added slowly to Part B. Once the addition is complete the mixture is allowed to continue for an additional 5 minutes to ensure entrainment of the liquid into the powder. The resulting composition is a free flowing, powder composition containing at least 85 wt % of Part A and 15 wt % Part B. The list of ingredients is set forth below in Table 1.

TABLE 1

| Part A | Description | Percent |
|---|---|---|
| 1 | CYCLOMETHICONE-PENTAMER | 10.0 |
| 2 | DIMETHICONE 50 CST | 3.6 |
| 3 | ISOSTEARYL PALMITATE | 0.4 |
| 4 | CYCLOMETH/DIMETH.COPOLYOL-92/8 | 7.8 |
| | (WT RATIO) | |
| 5 | CYCLOMETHICONE BLEND | 0.1 |
| 6 | TIT DIOX(20%)-CYCLOPENTA/ | 15.6 |
| | DIM.COP/AL.METH.DISP. | |
| 7 | DIMETHICONE COPOLYOL-HLB 4.5 | 0.7 |
| 8 | DIMETHICONOL | 0.3 |
| 9 | DIMETHICONE-POLYSILICONE-1/PET | 0.6 |
| 10 | DIMETHYL POLYSILOXANE | 1.6 |
| 11 | POE SORBITAN MONOSTEARATE | 0.9 |
| 12 | PROPYLENE GLYCOLO | 0.9 |
| | DICAPRYLATE | |
| 13 | CETYL RICINOLEATE | 0.9 |
| 14 | DEMINERALIZED WATER | 15.6 |
| 15 | DIPROPYLENE GLYCOL | 32.1 |
| 16 | GLYCERIN | 0.5 |
| 17 | PPG-5-CETETH-20 | 0.1 |
| 18 | PIGMENT | 7.8 |
| 19 | PRESERVATIVE | 0.4 |
| | TOTAL | 100.0 |
| | | Percent in |
| Part B | | Composition |
| 1 | LAURYL METHACRYLATE/ | 15 |
| | DIMETHACRYLATE CROSSPOLYMER | |
| | PARTICLE | |

TIT.DIOX.(20%)-CYCLOPENTA/DIM.COP./AL.METH.DISP. (a dispersion of titanium dioxide (with some alumina) in a blend of cyclopentasiloxane, PEG-10 dimethicone and methicone)

### Example 2

An emulsion having reactive components was prepared, adsorbed into polymer particles to produce a powder-to-liquid-to-powder composition.

The emulsion contains sodium sulfite and is made in a substantially similar manner as described above. 150 grams of Part B, lauryl methacrylate/dimethacrylate crosspolymer, is placed into a large stainless steel mixing bowl suitable or use with a Hobart Mixer (Model A-120) equipped with a wire-whisk mixing implement. Under low mixing, 850 grams of emulsion Part A is added slowly to Part B. Once the addition is complete, the mixture is allowed to continue for an additional 5 minutes to ensure entrainment. The resulting composition is a free flowing, powder composition containing at least 85 wt % of Part A and 15 wt % Part B.

Next, 150 grams of Part D, lauryl methacrylate/dimethacrylate crosspolymer particle, is placed into a large stainless steel mixing bowl suitable for use with a Hobart Mixer (Model A-120) equipped with a wire-whisk mixing implement. Under low mixing, 850 grams of the aqueous composition containing hydrogen peroxide, Part C, is added slowly to Part D. Once the addition is complete, the mixture is allowed to continue for an additional 5 minutes to ensure entrainment. The resulting composition is a free flowing, powder composition containing at least 85% of Part C and 15% Part D.

Finally, the powder-to-liquid-to-powder composition of Part A and Part B is mixed with the powder-to-liquid-to-powder composition of Part C and Part D in a 50/50 weight blend. Mixing is achieved by tumble mixing in a suitable container. The list of ingredients is set forth below in Table 2.

TABLE 2

| Part A | Description | Percent |
|---|---|---|
| 1 | CYCLOMETHICONE-PENTAMER | 10.8 |
| 2 | DIMETHICONE 50 CST | 4.0 |
| 3 | ISOSTEARYL PALMITATE | 0.5 |
| 4 | CYCLOMETH/DIMETH.COPOLYOL-92/8 | 8.5 |
| 5 | CYCLOMETHICONE BLEND | 0.1 |
| 6 | DIMETHICONOL | 0.4 |
| 7 | DIMETHICONE-POLYSILICONE-1/PET | 0.7 |
| 8 | DIMETHYL POLYSILOXANE | 1.8 |
| 9 | POE SORBITAN MONOSTEARATE | 0.9 |
| 10 | PROPYLENE GLYCOLO DICAPRYLATE | 0.9 |
| 11 | CETYL RICINOLEATE | 0.9 |
| 12 | DEMINERALIZED WATER | 30.0 |
| 13 | DIPROPYLENE GLYCOL | 34.6 |
| 14 | GLYCERIN | 0.6 |
| 15 | SODIUM SULFITE | 5.0 |
| 16 | PRESERVATIVE | 0.5 |
| | TOTAL | 100.0 |
| | | Percent in |
| Part B | | Composition |
| 1 | LAURYL METHACRYLATE/ | 15 |
| | DIMETHACRYLATE CROSSPOLYMER | |
| | PARTICLE | |
| Part C | Description | Percent |
| 12 | DEMINERALIZED WATER | 43.7 |
| 13 | DIPROPYLENE GLYCOL | 50.4 |
| 14 | GLYCERIN | 0.8 |
| 15 | HYDROGEN PEROXIDE | 4.4 |
| 16 | PRESERVATIVE | 0.7 |
| | TOTAL | 100.0 |
| | | Percent in |
| Part D | | Composition |
| 1 | LAURYL METHACRYLATE/ | 15 |
| | DIMETHACRYLATE CROSSPOLYMER | |
| | PARTICLE | |

### Example 3

A powder-to-liquid-to-powder composition have reactive components was prepared.

An emulsion containing glycolic acid is made in a substantially similar manner as described above. 150 grams of Part B, lauryl methacrylate/dimethacrylate crosspolymer particle, is placed into a large stainless steel mixing bowl suitable or use with a Hobart Mixer (Model A-120) equipped with a wire-whisk mixing implement. Under low mixing 850 grams of emulsion Part A is added slowly to Part B. Once the addition is complete the mixture is allowed to continue for an additional 5 minutes to ensure entrainment of the liquid into the powder. The resulting composition is a free flowing, powder composition containing at least 85 wt % of Part A and 15 wt % Part B.

Next, 150 grams of Part D, lauryl methacrylate/dimethacrylate crosspolymer particle, is placed into a large stainless steel mixing bowl suitable or use with a Hobart Mixer (Model A-120) equipped with a wire-whisk mixing implement. Under low mixing, 850 grams of the aqueous composition containing Carbopol 940, Part C, is added slowly to Part D. Once the addition is complete, the mixture is allowed to continue for an additional 5 minutes to ensure entrainment of the liquid into the powder. The resulting composition is a free flowing, powder composition containing at least 85% of Part C and 15% Part D.

Finally, the powder-to-liquid-to-powder composition of Part A and Part B is mixed with the powder-to-liquid-to-powder composition of Part C and Part D in a 50/50 blend. Mixing is achieved by tumble mixing in a suitable container. The list of ingredients is set forth below in Table 3.

TABLE 3

| Part A | Description | Percent |
|---|---|---|
| 1 | CYCLOMETHICONE-PENTAMER | 10.8 |
| 2 | DIMETHICONE 50 CST | 4.0 |
| 3 | ISOSTEARYL PALMITATE | 0.5 |
| 4 | CYCLOMETH/DIMETH.COPOLYOL-92/8 | 8.5 |
| | (WT RATIO) | |
| 5 | CYCLOMETHICONE BLEND | 0.1 |
| 6 | TIT DIOX (20%)-CYCLOPENTA/DIM.COP/ | 16.9 |
| | AL.METH.DISP | |
| 7 | DIMETHICONE COPOLYOL-HLB 4.5 | 0.8 |
| 8 | DIMETHICONOL | 0.4 |
| 9 | DIMETHICONE-POLYSILICONE-1/PET | 0.7 |
| 10 | DIMETHYL POLYSILOXANE | 1.8 |
| 11 | POE SORBITAN MONOSTEARATE | 0.9 |
| 12 | PROPYLENE GLYCOLO DICAPRYLATE | 0.9 |
| 13 | CETYL RICINOLEATE | 0.9 |
| 14 | DEMINERALIZED WATER | 16.9 |
| 15 | DIPROPYLENE GLYCOL | 29.8 |
| 16 | GLYCERIN | 0.6 |
| 17 | PPG-5-CETETH-20 | 0.1 |
| 18 | GLYCOLIC ACID | 5.0 |
| 19 | PRESERVATIVE | 0.5 |
| | TOTAL | 100.0 |
| | | Percent in |
| Part B | | Composition |
| 1 | LAURYL METHACRYLATE/ | 15 |
| | DIMETHACRYLATE CROSSPOLYMER | |
| | PARTICLE | |
| PART C | Description | Percent |
| 1 | DEMINERALIZED WATER | 67.58 |
| 2 | GLYCERIN | 7.50 |
| 3 | GLY ACRYLIC POLYMER PRES-MP/PP | 2.50 |
| 4 | DISODIUM EDTA-TECH.GRADE | 0.20 |
| 5 | CARBOPOL 940 | 1.00 |
| 6 | ACRYLATES/C10–30 ALK.ACRYL/TR-2 | 0.10 |
| 7 | C12–15 ALCOHOLS BENZOATE | 2.50 |
| 8 | C12–13 ALKYL LACTATE | 6.00 |
| 9 | BENZYL ALCOHOL | 1.00 |
| 10 | GLYCERYL MONOSTEARATE-NS EMUL | 0.75 |
| 11 | STEARETH-2 | 0.95 |
| 12 | PEG-40 STEARATE | 0.95 |
| 13 | BEHENYL ALCOHOL | 0.40 |
| 14 | PHYTOL | 1.00 |
| 15 | CYCLOMETHICONE-PENTAMER | 6.50 |
| 16 | SODIUM HYDROXIDE SOLUTION 50% | 0.67 |
| 17 | PRESERVATIVE | 0.40 |
| | TOTAL | 100.0 |
| | | Percent in |
| Part D | | Composition |
| 1 | LAURYL METHACRYLATE/ | 15 |
| | DIMETHACRYLATE CROSSPOLYMER | |
| | PARTICLE | |

GLY ACRYLIC POLYMER PRES-MP/PP (A glycerate-acrylic acid polymer containing water and propylene glycol)

### Example 4

A powder-to-liquid-to-powder composition was prepared from incompatible ingredients. The powder composition is formulated to deliver a Carbopol gel and high levels of pigments to a topical surface. The combination of a Carbopol gel and a high level of pigments is incompatible because Carbopol gels are known to flocculate pigments. The powder-to-liquid-to-powder composition maintains physical separation of the incompatible ingredients.

An emulsion containing 10 wt % pigments is prepared in a substantially similar manner as described above. 150 grams of Part B, lauryl methacrylate/dimethacrylate crosspolymer particle, is placed into a large stainless steel mixing bowl suitable or use with a Hobart Mixer (Model A-120) equipped with a wire-whisk mixing implement. Under low mixing, 850 grams of emulsion Part A is added slowly to Part B. Once the addition is complete, the mixture is allowed to continue for an additional 5 minutes to ensure entrainment of the liquid into the powder. The resulting composition is a free flowing, powder composition containing at least 85 wt % of Part A and 15 wt % Part B.

Next, 150 grams of Part D, lauryl methacrylate/dimethacrylate crosspolymer particle, is placed into a large stainless steel mixing bowl suitable or use with a Hobart Mixer (Model A-120) equipped with a wire-whisk mixing implement. Under low mixing, 850 grams of the aqueous composition containing Carbopol 940, Part C, is added slowly to Part D. Once the addition is complete the mixture is allowed to continue for an additional 5 minutes to ensure entrainment of the liquid into the powder. The resulting composition is a free flowing, powder composition containing at least 85% of Part C and 15% Part D.

Finally, the powder-to-liquid-to-powder composition comprised of Part A and Part B is mixed with the powder-to-liquid-to-powder composition comprised of Part C and Part D in a 50/50 blend. Mixing is achieved by tumble mixing in a suitable container. The list of ingredients is set forth in Table 4.

TABLE 4

| Part A | Description | Percent |
|---|---|---|
| 1 | CYCLOMETHICONE-PENTAMER | 10.8 |
| 2 | DIMETHICONE 50 CST | 4.0 |
| 3 | ISOSTEARYL PALMITATE(*RI*) | 0.5 |
| 4 | CYCLOMETH/DIMETH.COPOLYOL-92/8 | 8.5 |
| | (WT RATIO) | |
| 5 | CYCLOMETHICONE BLEND | 0.1 |
| 6 | TIT DIOX (20%)-CYCLOPENTA/DIM.COP/ | 11.9 |
| | AL.METH.DISP. | |
| 7 | DIMETHICONE COPOLYOL-HLB 4.5 | 0.8 |
| 8 | DIMETHICONOL | 0.4 |
| 9 | DIMETHICONE-POLYSILICONE-1/PET | 0.7 |
| 10 | DIMETHYL POLYSILOXANE | 1.8 |
| 11 | POE SORBITAN MONOSTEARATE | 0.9 |
| 12 | PROPYLENE GLYCOLO DICAPRYLATE | 0.9 |
| 13 | CETYL RICINOLEATE | 0.9 |
| 14 | DEMINERALIZED WATER | 16.9 |
| 15 | DIPROPYLENE GLYCOL | 29.8 |
| 16 | GLYCERIN | 0.6 |
| 17 | PPG-5-CETETH-20 | 0.1 |
| 18 | PIGMENTS | 10.0 |
| 19 | PRESERVATIVE | 0.5 |
| | TOTAL | 100.0 |
| | | Percent in |
| Part B | | Composition |
| 1 | LAURYL METHACRYLATE/ | 15 |
| | DIMETHACRYLATE CROSSPOLYMER | |
| | PARTICLE | |
| PART C | Description | Percent |
| 1 | DEMINERALIZED WATER | 67.58 |
| 2 | GLYCERIN | 7.50 |
| 3 | GLY ACRYLIC POLYMER PRES-MP/PP | 2.50 |
| 4 | DISODIUM EDTA-TECH.GRADE | 0.20 |
| 5 | CARBOPOL 940 | 1.00 |
| 6 | ACRYLATES/C10–30 ALK.ACRYL/TR-2 | 0.10 |
| 7 | C12–15 ALCOHOLS BENZOATE | 2.50 |
| 8 | C12–13 ALKYL LACTATE | 6.00 |
| 9 | BENZYL ALCOHOL | 1.00 |
| 10 | GLYCERYL MONOSTEARATE-NS EMUL | 0.75 |
| 11 | STEARETH-2 | 0.95 |
| 12 | PEG-40 STEARATE | 0.95 |
| 13 | BEHENYL ALCOHOL | 0.40 |
| 14 | PHYTOL | 1.00 |
| 15 | CYCLOMETHICONE-PENTAMER | 6.50 |
| 16 | SODIUM HYDROXIDE SOLUTION 50% | 0.67 |
| 17 | PRESERVATIVE | 0.40 |
| | TOTAL | 100.0 |
| | | Percent in |
| Part D | | Composition |
| 1 | LAURYL METHACRYLATE/ | 15 |
| | DIMETHACRYLATE CROSSPOLYMER | |
| | PARTICLE | |

It should be understood that the foregoing description is only illustrative of the present disclosure. Various alternatives and modifications can be devised by those skilled in the art without departing from the present disclosure. Accordingly, the present disclosure is intended to embrace all such alternatives, modifications and variances that fall within the scope of the appended claims.

What is claimed is:

1. A method of imparting a cosmetic or therapeutic benefit to a topical surface, comprising:
a) providing a polymer powder having a plurality of substantially nonporous polymer particles with a number average particles size of about 0.1 to about 0.5 microns in diameter;
b) providing a liquid cosmetic or therapeutic composition in the form selected from the group consisting of a solution, dispersion, suspension, and an emulsion, wherein the liquid composition has one or more cosmetic or therapeutic actives therein and is metastable;
c) contacting the polymer powder with the liquid composition so that the liquid composition is entrained so as to form an impregnated powder; and
d) rubbing the impregnated powder on the topical surface such that at least a substantial portion of the liquid composition is released from the impregnated powder at the topical surface.

2. The method of claim 1, wherein the liquid composition is an emulsion.

3. A method of imparting a cosmetic or therapeutic benefit to a topical surface, comprising:
a) providing a polymer powder having a plurality of substantially nonporous polymer particles with a number average particles size of about 0.1 to about 0.5 microns in diameter;
b) providing a liquid cosmetic or therapeutic composition selected from the group consisting of an water-in-oil emulsion, water-in-silicone emulsion, and a water-in-wax emulsion, wherein the liquid composition has one or more cosmetic or therapeutic actives; and
c) contacting the polymer powder with the liquid composition so that the liquid composition is entrained so as to form an impregnated powder; and
d) rubbing the impregnated powder on the topical surface such that at least a substantial portion of the liquid composition is released from the impregnated powder at the topical surface.

4. The method of claim 3, wherein the liquid composition is water-in-silicone emulsion.

5. A method of imparting a cosmetic or therapeutic benefit to a topical surface, comprising:
a) providing a polymer powder having a plurality of substantially nonporous polymer particles with a number average particles size of about 0.1 to about 0.5 microns in diameter;
b) providing two or more reactive components in liquid form therein that are reactable with each other to form one or more cosmetic or therapeutic actives or produce one or more cosmetic or therapeutic effects;
c) contacting each of the two or more reactive components in liquid form with a batch polymer powder to form two or more impregnated batch polymer powders wherein each reactive component is entrained in an impregnated batch powder;
d) admixing the two or more impregnated batch polymer powders to form a combined impregnated polymer powder; and
e) rubbing the combined impregnated powder on the topical surface such that at least a substantial portion of the two or more reactive components are released from the combined impregnated powder and are allowed to react to produce the one or more cosmetic or therapeutic actives or the one or more cosmetic or therapeutic effects at the topical surface.

6. A method of imparting a cosmetic or therapeutic benefit to a topical surface, comprising:
a) providing a polymer powder having a plurality of substantially nonporous polymer particles with a number average particles size of about 0.1 to about 0.5 microns in diameter;
b) providing two or more incompatible cosmetic or therapeutic components in liquid form;
c) contacting each of the two or more incompatible components in liquid form with a batch polymer powder to form two or more impregnated batch polymer powders, wherein each incompatible component is entrained in an impregnated batch powder;
d) admixing the two or more impregnated batch polymer powders to form a combined impregnated polymer powder; and
e) rubbing the combined impregnated powder on the topical surface such that at least a substantial portion of the two or more incompatible components are released at the topical surface.

7. A process for making an impregnated polymer powder for imparting a cosmetic or therapeutic benefit to a topical surface, comprising:
a) providing a polymer powder having a plurality of substantially nonporous polymer particles having a number average particles size of about 0.1 to about 0.5 microns in diameter;
b) providing a liquid cosmetic or therapeutic composition in the form selected from the group consisting of a solution, dispersion, suspension, and an emulsion, wherein the liquid composition has one or more cosmetic or therapeutic actives therein and is metastable; and
c) contacting the polymer powder with the liquid composition so that the liquid composition is entrained so as to form an impregnated powder.

8. The process of claim 7, wherein the liquid composition is an emulsion.

9. A process for making an impregnated polymer powder for imparting a cosmetic or therapeutic benefit to a topical surface, comprising:
a) providing a polymer powder having a plurality of substantially nonporous polymer particles with a number average particles size of about 0.1 to about 0.5 microns in diameter;
b) providing a liquid cosmetic or therapeutic composition selected from the group consisting of an water-in-oil emulsion, water-in-silicone emulsion, and a water-in-wax emulsion, wherein the liquid composition has one or more cosmetic or therapeutic actives; and
c) contacting the polymer powder with the liquid composition so that the liquid composition is entrained so as to form an impregnated powder.

10. The process of claim 9, wherein the liquid composition is water-in-silicone emulsion.

11. A process for making an impregnated polymer powder for imparting a cosmetic or therapeutic benefit to a topical surface, comprising:
a) providing a polymer powder having a plurality of substantially nonporous polymer particles with a number average particles size of about 0.1 to about 0.5 microns in diameter;
b) providing two or more reactive components in liquid form therein that are reactable with each other to form one or more cosmetic or therapeutic actives or produce one or more cosmetic or therapeutic effects;
c) contacting each of the two or more reactive components in liquid form with a batch polymer powder to form two or more impregnated batch polymer powders wherein each reactive component is entrained in an impregnated batch powder; and
d) admixing the two or more impregnated batch polymer powders to form a combined impregnated polymer powder.

12. A process for making an impregnated polymer powder for imparting a cosmetic or therapeutic benefit to a topical surface, comprising:
a) providing a polymer powder having a plurality of substantially nonporous polymer particles with a number average particles size of about 0.1 to about 0.5 microns in diameter;
b) providing two or more incompatible cosmetic or therapeutic components in liquid form;
c) contacting each of the two or more incompatible components in liquid form with a batch polymer powder to form two or more impregnated batch polymer powders wherein each incompatible component is entrained in an impregnated batch powder; and
d) admixing the two or more impregnated batch polymer powders to form a combined impregnated polymer powder.
